(19) United States
(12) Patent Application Publication
(10) Pub. No.: US 2010/0152579 A1
(43) Pub. Date: Jun. 17, 2010

(54) PORTABLE ULTRASONIC DEVICE AND ALGORITHMS FOR DIAGNOSIS OF DENTAL CARIES

(75) Inventors: Wei LIN, Stony Brook, NY (US); Mark S. Wolff, E. Moriches, NY (US); Anilkumar R. Dhundale, Stony Brook, NY (US)

Correspondence Address: THE FARRELL LAW FIRM, LLP, 290 Broadhollow Road, Suite 210E, Melville, NY, 11747, US

(73) Assignee: The Research Foundation of State University of New York, Albany, NY (US)

(21) Appl. No.: 12/618,352

(22) Filed: Nov. 13, 2009

Related U.S. Application Data
(63) Continuation-in-part of application No. 11/374,737, filed on Mar. 14, 2006, now abandoned.

(51) Int. Cl. A61B 8/14

(52) U.S. Cl. 600/437

(57) ABSTRACT

Provided is an apparatus and method for the detection of dental caries. The apparatus generates longitudinal ultrasound waves that may be applied to any accessible surface of a tooth. The reflected ultrasound pulse echoes are collected and correlated with the incident pulse. The ultrasound pulse echoes from the front and rear surface of a dental cavity may be distinguished from other echoes, and provided in visual display to inform as to the size and location of the cavity.

### PRIORITY

This application is a continuation-in-part of application Ser. No. 11/374,737, filed Mar. 14, 2004, the contents of which are hereby incorporated by reference.

### BACKGROUND

1. Field of the Invention

The present invention relates to an apparatus and method for the ultrasonic detection of dental caries using longitudinal ultrasonic waves.

2. Description of Related Art

X-ray technology is routinely utilized to examine for dental caries, i.e. dental decay. Dental radiographs that are performed to detect dental caries, show density differences in tooth structure caused by loss of calcium. Dental radiographs, however, can only be utilized to look for caries on the two accessible side surfaces of teeth. The remaining structures, particularly the occlusal, i.e. biting, surface, frequently develop considerably large carious lesions that remain undetectable to physical examination with the dental probe or by radiographic examination. These devices typically fail to detect a cavity until an advanced stage, and therefore are not good for early detection and treatment. A dental radiograph is radioactive, and therefore carries some degree of risk. Dental radiography is also expensive and non-portable.

Dental cavities most commonly develop on a tooth's biting surface. However, most caries have small entry openings and a much larger void can exist under the biting surface. These cavities cannot be easily found with the commonly used dental probes or X-rays. An existing device, DIAGNOdent, KaVo and KaVo America Corp. (www.kavo.com, www.kavousa.com) utilizes a colorimetric approach for detecting cavities. Although such conventional device improves early detection, such device is limited in performance for early detection, for example, because of non-uniformities in tooth color that create false signals or mask real signals.

Ultrasonic detection of dental caries has been attempted as a method to overcome the above-mentioned deficiencies associated with conventional techniques for detecting caries. U.S. Pat. No. 6,162,177 to Bab et al., the contents of which is incorporated by reference, presented a device and method for the ultrasonic detection of smooth surface lesions on tooth crown surfaces such as caries and tooth crown surface cracks on a tooth crown surface. Bab et al. provides an ultrasonic surface wave transmitter/receiver, capable of transmitting an ultrasonic surface wave along a tooth crown surface. Surface lesions exposed to ultrasonic surface waves create ultrasonic surface wave reflections, which may be received at the transmitter/receiver, thereby detecting the presence of a cavity. Ultrasonic surface waves are capable of only limited penetration into the tooth and therefore, are unable to detect deeply penetrating caries or to determine the size of the caries.

U.S. Pat. No. 7,175,599 to Hynynen et al., the contents of which is incorporated by reference, presented a device that can detect soft tissues inside skull using trans-skull ultrasound. Hynynen employs a shear wave to minimize attenuation of the ultrasound wave in the skull and to maximize acoustic energy reaching the soft tissue for the measurement of blood flow in the brain. Hynynen requires that the ultrasound beam enter the skull in a specific angle, to convert a longitudinal wave to shear wave in the skull and for conversion back into a longitudinal wave when exiting the opposite side of the skull bone and entering the brain. However, Hynynen is not usable to detect dental cavities, at least because Hynynen fails to minimize a bone surface boundary effect of ultrasound waves and cannot efficiently determine cavity size. In contrast, the present invention utilizes reflections from bone surfaces to determine cavity size.

The present invention overcomes the shortcomings of conventional systems by providing an apparatus and algorithms for measuring and displaying the size of dental caries, by use of the tooth penetrating capacity of longitudinal ultrasound waves. The present invention utilizes signal processing to extract echoes from a received ultrasound signal for improved cavity size measurement, without decay due to overlap of echoes from the front and back ends of the cavity, which is common due to the small size of the cavity being measured in teeth.

### SUMMARY OF INVENTION

The present invention addresses the above problems and/or disadvantages and provides at least the advantages described below. In a preferred embodiment of the present invention, a portable apparatus is provided for the detection of dental caries, in which a transducer is positioned adjacent to a tooth; an ultrasound pulse is transmitted as a longitudinal wave into the tooth; first and second wave reflections are received; a time difference between the first and second reflections is determined; the first and second reflections are correlated with the transmitted ultrasound pulse; and a location and size of a dental cavity is determined based on correlation of a position of the transducer, the transmitted ultrasound pulse, and the time difference between the first and second reflections. In a preferred embodiment, an echo detection unit is provided for use by phase loop lock algorithm for echo detection.

### BRIEF DESCRIPTION OF THE DRAWINGS

The above and other aspects and advantages of the present invention will be more clearly understood from the following detailed description taken in conjunction with the accompanying drawings in which:

FIG. 1 is a block diagram of an ultrasonic apparatus for the detection of dental caries according to a first embodiment of the present invention; and

FIGS. 2(a) and 2(b) are results of a time shifting frequency analysis;

FIG. 3 is a Morlet wavelet showing;

FIG. 4 is a chart of phase alignment using frequency sweep signals to obtain sub-millimeter resolution; and

FIG. 5 is a chart showing overlapping echoes of the phase lock algorithm.

### DETAILED DESCRIPTION OF THE PREFERRED EMBODIMENTS

Hereinafter, preferred embodiments of the present invention will be described with reference to the accompanying drawings. It should be noted that the similar components may be designated by similar reference numerals although they are illustrated in different drawings. Also, in the following description, a detailed description of known functions and configurations incorporated herein will be omitted when it may obscure the subject matter of the present invention.

A novel approach to detect dental caries is provided using one or more of the algorithms described below along with a modulated or an un-modulated ultrasonic wave transmitted through a tooth that is under examination. When an ultrasound wave enters the tooth, wave reflections occur at density change interfaces, such as the enamel-dentin interface, as well as at the dentin-pulp interface. These reflections, i.e. displacements in relation to time, make it possible to measure the thickness of the enamel and dentin, which are important indexes of tooth quality. In addition, when a dental cavity occurs inside a tooth, the cavity, because it is fluid filled and therefore of different density in comparison with surrounding tissue, will cause a change in the pattern of ultrasound reflection waves that will be detectable and quantifiable. This present invention uses an ultrasound wave to identify and estimate the size of a dental cavity as small as 1/10 of a millimeter in diameter.

Longitudinal ultrasound pulses, at a preferred center frequency of 10 MHz, are applied by the transducer at an accessible tooth surface. Echoes of the incident ultrasound pulse reflect from internal tooth density interfaces including the front and back of the cavity. The ultrasound pulses are converted to echo electric pulses by the transducer, switched for collection and amplification by the ultrasound receiver, and the echoes from the front and rear surfaces of the dental caries are correlated by the echo detection unit and controller to distinguish them from the other echoes from other surfaces and interfaces of the tooth.

Conduction of ultrasound wave is weakened due to air present in the cavity. Air is not a good wave conductor. A weak signal will penetrate the air, travel across the cavity and reflect off the opposite solid dentin or enamel surface. If fluid is present in a cavity, much better conductance will be obtained and the wave reflected from the front and back end of the cavity will be detected as a stronger signal. In a preferred embodiment of the present invention, the patient performs a simple mouthwash with water prior to scanning should fill all cavities with fluid.

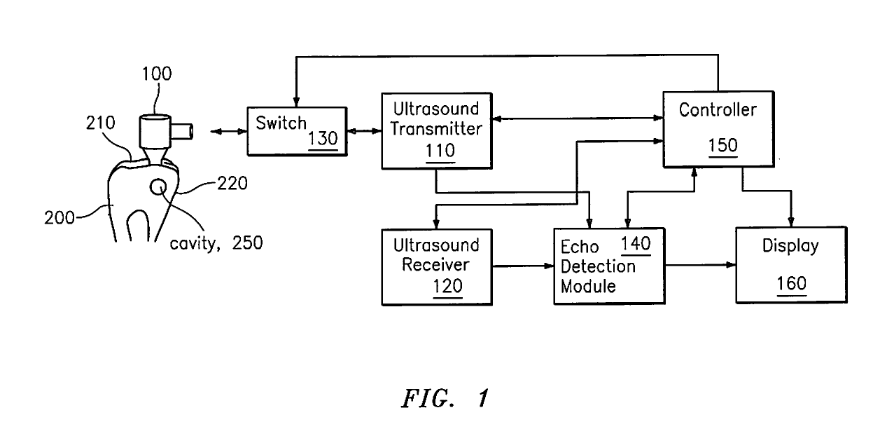

An apparatus according to the first embodiment of the invention is shown in FIG. 1. A dental caries detection apparatus 10 consists of a transducer 100, an ultrasound transmitter 110, an ultrasound receiver 120, a switch 130, an echo detection module 140, a controller 150, and a display 160. The dental caries detection apparatus 10 is applied to a tooth 200 to sense a dental cavity 250. The ultrasound transmitter 110 generates an electric pulse in the form of a short un-modulated pulse or a modulated signal to drive the transducer 100 to apply the ultrasound pulse to the tooth 200. The controller 150 determines a rate and energy of the pulse or signal. In a modulation mode, the controller also defines the signal format, e.g. frequency sweep signal with known duration and start/end frequencies. Ultrasound waves with a frequency of 1 MHz to 20 MHz are preferably used, with 10 MHz as the preferred frequency due to superior signal correlating capability in this range.

The transducer 100 converts the electric pulse or modulated ultrasound signal to an ultrasound pulse to either the occlusal surface 210 or a side surface 220 of the tooth 200. The transducer 100 is in a preferred embodiment is a single element transducer with diameter up to 5 mm, preferably 1 mm for optimal spatial resolution.

The transducer 100 is in a preferred embodiment a two-dimensional transducer array with N×N elements, with a preferred value of N=3. The maximum value of N is determined by the minimum achievable size of the transducer element. Each element serves as an individual ultrasound transducer, which has the capability of transmitting and receiving ultrasound signals. The activation of the transducer 100 is controlled by the switch 130 and the controller 150. By activating each element, the location of the ultrasound signal reflection and hence the cavity surface can be identified.

Alternatively, the transducer 100 can be a micro mechanical device with one transducer element mounting on a two-dimensional micro-stage. The movement of the stage is driven by step motors and micro positioning devices, as known in the art. The control signal to the moving stage is sent from the controller 150 via the switch 130 to the transducer 100. The location of the transducer 100 is determined by the controlled positioning of the two-dimensional stage.

The switch 130, under the control of the controller 150, serves as a router, directing the electric pulse from the ultrasound transmitter 110 to the transducer 100 where it is transformed into an ultrasound pulse. The switching function is controlled by the controller 150. The ultrasound waves pass from the transducer 100 through one of the occlusal surface 210 and the side surface 220 into the tooth 200 where some of the energy of the incident wave is reflected, as ultrasound echoes, by the internal structures of the tooth 200. The signal reflection will be discussed later in more detail. The transducer 100 converts the ultrasound echoes back into electric pulses. The switch 130, under the control of the controller 150, directs the echo electric pulses to the ultrasound receiver 120.

In a transducer array mode, the switch 130 multiplexes the ultrasound electric pulses to the individual transducer element. The controller 150 provides address signals to the switch 130 to determine which element is activated. In the micro-stage mode, the controller 150 sends position pulses to the micro-positioning device via the switch 130 to move the transducer 100 to a known position.

The ultrasound receiver 120 collects and amplifies the received electric pulses from the transducer for analysis. In the preferred embodiment, the gain of the receiver is programmable in a range of 0 to 60 dB. In order to compensate for ultrasound energy attenuation in the tooth, the gain is controlled according to the time delay, i.e. the gain is increased for the signal coming from deep inside the tooth.

The echo detection module 140 receives the amplified received electric pulses and the electric pulses from the ultrasound transmitter 110 and identifies the location at which the echoes were generated from the different locations in the tooth.

Described herein are four algorithms available for use in echo detection. A Time Shifting Frequency Analysis (TSFA) technique localizes echoes from the cavity and derives the cavity size based on frequency harmonics resulting from acoustic reflections with the cavity. The TSFA method applies to an un-modulated ultrasound pulse. A wavelet algorithm decomposes the ultrasound signal into a series of wavelets spread out in the time domain. If a wavelet (Morlet wavelet) close to the waveform of an ultrasound pulse is selected, time locations of the echoes are determined from the peaks of the wavelet coefficients. If the ultrasound pulse has the same shape of Morlet wavelet, improved analysis accuracy is achieved using an un-modulated ultrasound pulse approximation. A phase alignment algorithm uses modulated ultrasound signals, i.e. frequency sweep signal. By aligning the phase of all frequency components with the echo, the frequency sweep signal becomes a short pulse, achieving a resolution on the order of 0.1 mm to detect the cavity. A phase lock algorithm detects echoes based on the phase of the echoes, preferably using a tone burst signal in the form of modulated ultrasound. In the following description, one or more algorithms are utilized to enhance measurement of the location and size of the cavity.

The TSFA algorithm determines a localized frequency spectrum of the received longitudinal ultrasound wave as the signal changes over time. The TSFA algorithm is designed for an un-modulated ultrasound pulse. The TSFA algorithm applies a time windowing function to the signal before Fourier transformation and calculates the frequency spectrum pertaining to that section of signal. As the window slides across the signal in the time domain, the TSFA algorithm generates a two-dimensional representation of the signal in both the time domain and the frequency domain. In Equation (1), x(t) is the signal and w(t) is the windowing function.

$$X ( ω , τ ) = {\int }_{- ∞}^{∞} x ( t ) w ( t - τ ) {e}^{- jω t} d t \qquad ( 1 )$$

The TSFA recognizes that ultrasound pulses received from the location of cavity are distinct in time domain. The shifting of the window function in the time domain localizes the pulses received from the cavity boundary, and a special focus is placed on searching repetitive harmonic peaks in the frequency spectrum and associated frequencies. Peak frequencies are used to calculate the lesion size, according to Equation (2), in which c is the ultrasound velocity in the cavity and Δf is the difference between the peak frequencies:

$$w = \frac{c}{2 Δ f} \qquad ( 2 )$$

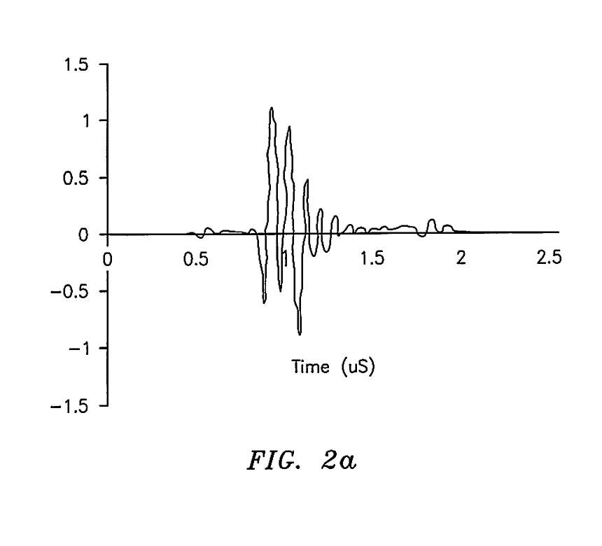

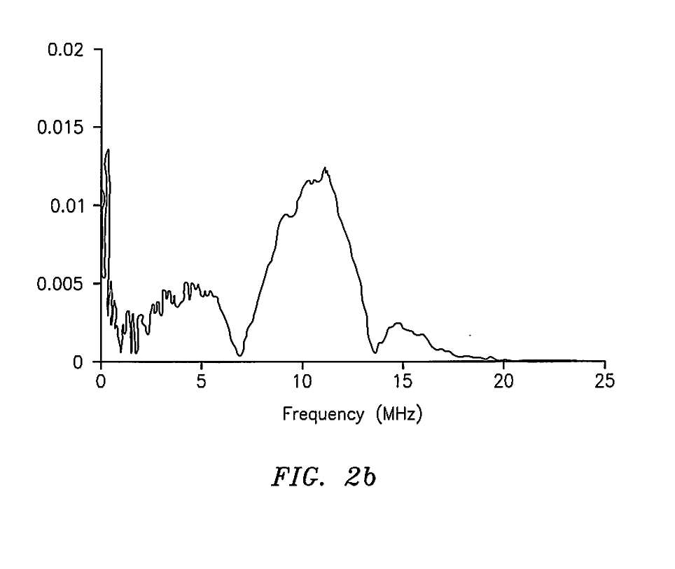

FIG. 2(a) shows ultrasound pulses received from a simulated cavity and FIG. 2(b) shows associated pulses from the TSFA algorithm measured of a 0.11 mm crack filled with water. The spectrum shows harmonic peaks that correlate with the crack size. Table 1 shows ultrasound results from tests performed on samples of three different crack sizes.

TABLE 1

| | | | |
|---|---|---|---|
| Actual Crack Size | 0.11 mm | 0.22 mm | 0.33 mm |
| Estimated Size | 0.11 mm | 0.23 mm | 0.35 mm |

The wavelet analysis is another tool for time frequency analysis using a multi-scaled approach. In this algorithm, the wavelet is a fast-decaying oscillating waveform of finite duration. A wavelet transform decomposes the signal into a series of scaled and time delayed wavelets. Equation (3) shows the wavelet transform, where ψ(t) is the wavelet function. Equation (3) yields a coefficient w(a,b) representing the magnitude of the wavelet of the time scale of a at the time point of b.

$$w ( a , b ) = \int x ( t ) \frac{1}{\sqrt{a}} ψ \overline{( \frac{t - b}{a} )} d t \qquad ( 3 )$$

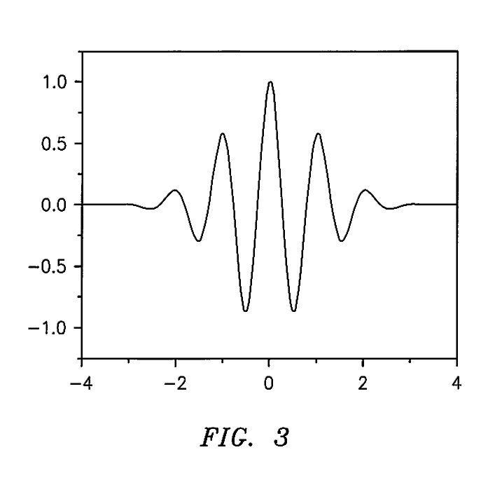

The received ultrasound pulses are composed of a series of echoes of the similar waveform with different delays from cavity. FIG. 3 shows a Morlet wavelet, which is utilized for modeling in the analysis utilizing the Morlet shape that is close to the ultrasound pulse. The analysis is performed on the search of the maximum wavelet coefficients and corresponding time points, which represents the time locations of the ultrasound echo from the boundary of the cavity. The time points are used to calculate the size and location of the cavity, using Equation (4):

$$w = \frac{1}{2} Δ t \times c \qquad ( 4 )$$

where w is the size of the cavity and Δt is the time difference between the ultrasound pulses from the front and back of the cavity wall, and C is the ultrasound wave velocity in the cavity; and Equation (5):

$$d = \frac{1}{2} t \times c \qquad ( 5 )$$

where d is the location of the cavity from surface and t is the time of round trip from tooth surface to the front cavity wall, and C is the ultrasound wave velocity in the cavity.

The Wavelet algorithm is applied to un-modulated ultrasound pulse or modulated ultrasound pulse having a waveform is shaped as the Morlet wavelet. Improved accuracy is achieved by use of the Morlet shaped ultrasound signal to eliminate approximation error resulting from use of an un-modulated ultrasound pulse.

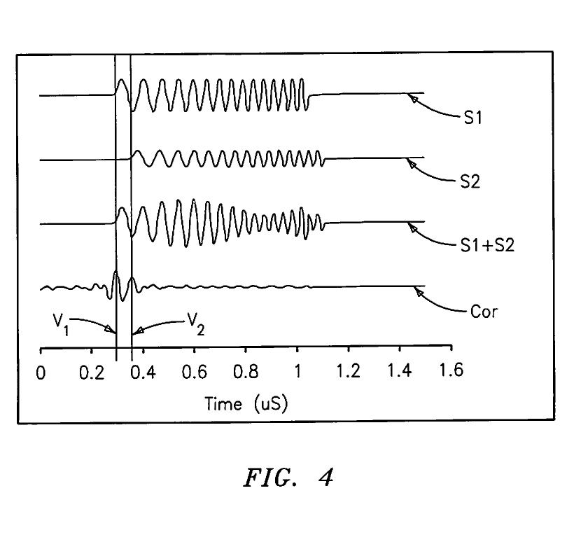

The phase alignment algorithm uses a frequency sweep signal. In this algorithm, the ultrasound signal used for cavity detection is a modulated signal, such as a frequency sweep signal. FIG. 4 shows use of a frequency sweep signal to identify the cavity size, which cannot be achieved by simple pulse signal. In FIG. 4, echoes S1 and S2 are received from the front and back surfaces of a cavity with the delay indicated by vertical cursors (V1 and V2). Both echoes were overlapped and otherwise are not distinguishable in the time domain. See “S1+S2” of FIG. 4. However, upon correlation to the original frequency sweep signal that generated echoes S1 and S2, the peaks of the correlation output (“Cor”) correspond to the locations of both echoes (S1 and S2). The correlation aligns the phases of all frequency components and converts the frequency sweep signals into a narrow pulse.

The data shown in FIG. 4 was obtained using a start frequency of the frequency sweep signal of 10 MHz, a time delay of approximately 0.07 μS between two echoes, less than one wavelength (0.15 mm in water) in space. The frequency sweep signal shown in FIG. 4 can be used to achieve sub-wavelength resolution suitable for cavity detection, and FIG. 4 shows phase alignment using frequency sweep signals to obtain sub-millimeter resolution.

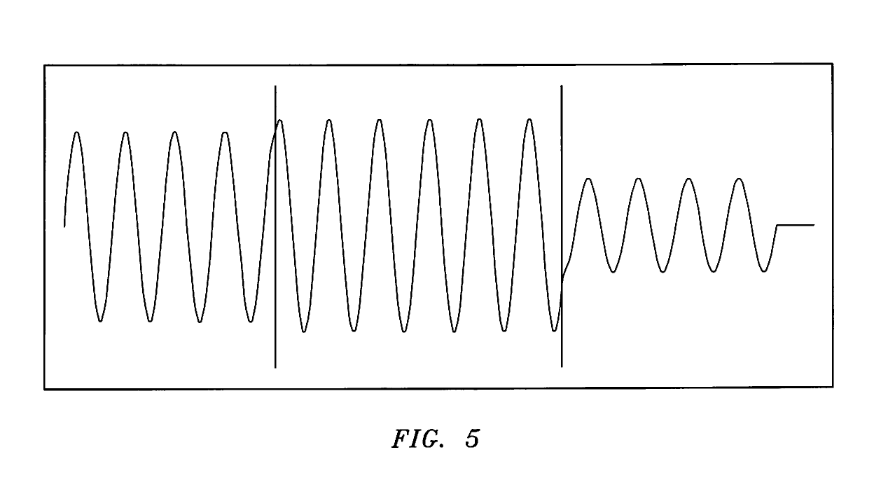

In the phase lock algorithm, phase locking is utilized to locate the ultrasound echoes from the dental cavity. As shown in FIG. 5, two tone burst signals, as echoes from the cavity, overlap. Phase change at each overlap points is indicated in FIG. 5 by the vertical lines. The phase locking algorithm detects the phase changes.

The phase lock algorithm tracks and can dynamically detects the phase of sinusoidal signals. The phase lock algorithm simulates the measured sinusoidal signal d(T) at time T, e.g. the tone burst signal, with a linear combination of known sine and cosine wave yₚ(T), pursuant to Equation (6):

yₚ(T)=w₁(T)·sin(2πfT)+w₂(T)cos(2πfT) (6)

where w₁(T) and w₂(T) are dynamically adjusted coefficients, and f is a specified frequency. The adjustment of the coefficients is performed at each time step based on the least square criterion of the prediction error e(T), or Equation (7):

Min(Σ(d(T)−yₚ(T))²) (7)

When the above criterion is reached, the sinusoidal signal is considered tracked by yₚ(T). The amplitude Amp(T) and phase φ(T) of the signal can be calculated from w₁(T) and w₂(T) of Equation (8):

$$Amp ( T ) = \sqrt{{w}_{1}^{2} ( T ) + {w}_{2}^{2} ( T )} \\ φ ( T ) = - {tan}^{- 1} ( \frac{{w}_{2} ( T )}{{w}_{1} ( T )} ) \qquad ( 8 )$$

where φ(T) is the phase lag in reference to sin(2πfT). The phase tracking method uses the least square algorithm for the transversal adaptive filter to calculate w₁(T) and w₂(T).

When the ultrasound signal is in a tone burst format, a series of sinusoidal cycles of a specific frequency, phase locking can detect the phase of the ultrasound echo signals with respect to the referenced signal, which bears the same frequency as the tone burst. Echoes from different locations within the tooth, such as the cavity wall, have different phase values. Those echoes are usually overlapped, as shown in FIG. 5. The phase locking algorithm tracks the phase of the received ultrasound signal in real time. If there is a sudden change in phase, a determination is made that echo overlap has occurred at that specific time point. Therefore, the time of phase change from the phase lock algorithm is in a preferred embodiment used to determine the location and size of the cavity, based on Equations (4) and (5). The detected phase value can be converted to a time location of the received ultrasound signals.

When the ultrasound waves impinge on a tooth surface, some of the energy of the incident wave is reflected in the form of echoes. Prominent echoes are returned from the edges of the cavity, in addition to those from the tooth surface, the enamel-dentin interface, and the dentin-pulp interface. The echoes may be shifted in magnitude, direction and time due to the complex internal structure of the tooth and the cavity. The location and size of the cavity can be obtained according to the location of the echoes from the abnormal sites using the abovementioned techniques.

When an ultrasound pulse is transmitted into a tooth structure, first and second reflections are received from a dental cavity. The first reflection, i.e. pulse, represents an incident ultrasound wave. The pulses of the first and second reflections from the front and back of the cavity, have a time difference (dt) therebetween. The size of the cavity may be calculated based on the time difference between the two pulses using simple time calculations, such as s=c*dt/2 where s is the size of the cavity, c is a known speed of the ultrasound pulse in a cavity in the fluid filled medium of a cavity. However, echoes often overlap, making cavity detection difficult. The algorithms described herein extract echo locations from the received ultrasound and determine the location and size of the cavity.

The controller 150 coordinates the generation and receipt of the ultrasound pulse, the performance and analysis of the collected data, and the generation of a report based on the evaluation.

A micro mechanical transducer is used in one embodiment, with one transducer element mounted on a two dimensional micro-stage, and control signals are sent from the controller 150 to the micro-stage as the micro-stage moves via the switch 130 to the transducer 100. The controller 150 determines the rate and energy of the ultrasound signals generated in the ultrasound transmitter 110. In the modulation mode, the controller 150 also defines a signal format, e.g. frequency sweep signal with known duration and start/end frequencies.

The raw image obtained from the ultrasound signal can be displayed on a display 160 to help the physician position the probe, along with an analysis result. The display preferably includes a cavity dimension calculated by the controller 150. Further, measurement of the cavity is preferably performed from results obtained with the transducer positioned at several different locations on the external surface of the tooth, to provide a three-dimensional view of the cavity, with a display of major internal and external surfaces of the affected tooth.

While the invention has been shown and described with reference to a certain preferred embodiment thereof, it will be understood by those skilled in the art that various changes in form and details may be made therein without departing from the spirit and scope of the invention as defined in the appended claims.

What is claimed:

1. A method for detecting dental caries, the method comprising:
positioning a transducer adjacent to a tooth;
transmitting an ultrasound pulse as a longitudinal wave into the tooth;
receiving first and second reflections;
detecting a time difference between the first and second reflections;
correlating the first and second reflections with the transmitted ultrasound pulse; and
determining a location and size of a dental cavity based on correlation of a position of the transducer, the transmitted ultrasound pulse, and the time difference between the first and second reflections.

2. The method of claim 1, wherein correlating of the first and second reflections with the transmitted ultrasound pulse includes using cross correlating techniques.

3. The method of claim 2, wherein the cross correlating techniques include a phase loop locking technique performed by an echo detection unit.

4. The method of claim 2, wherein the first and second reflections are received ultrasound pulse echoes representing a front surface of the dental cavity and a back surface of the dental cavity, respectively.

5. The method of claim 4, wherein a size of the dental cavity is determined based on:
s=c*dt/2,
where s is dental cavity size, c is a known speed of the ultrasound pulse in a fluid medium filled in a cavity, and dt is the time difference between the first and second reflections.

6. The method of claim 1, wherein the dental cavity is filled with a fluid by use of mouthwash before the positioning the transducer.

7. The method of claim 1, wherein the ultrasound pulses are modulated pulses generated in a frequency range between 1 and 20 MHz.

8. The method of claim 1, wherein the transducer is positioned on an occlusal surface of the tooth.

9. The method of claim 1, wherein the transducer is positioned on a side surface of the tooth.
